(19) United States
(12) Patent Application Publication
(10) Pub. No.: US 2023/0211062 A1
(43) Pub. Date: Jul. 6, 2023

(54) HEMODIALYSIS SYSTEM WITH DIALYSATE RECYCLING

(71) Applicant: KING FAISAL UNIVERSITY, AL-AHSA (SA)

(72) Inventors: SARAH MOHAMMED ALQATTAN, HUFUF (SA); MARWA ABDULLATIF ALJAFFRI, HUFUF (SA); FATIMAH HASSAN ALBAHRANI, HUFUF (SA); GHADI FARIS ALQAHTANI, HUFUF (SA); SHOUQ ABDULRAUF ALBUSHAIER, HUFUF (SA); ZAINAB AHMED ALAWADH, ALOMRAN (SA)

(21) Appl. No.: 17/568,680

(22) Filed: Jan. 4, 2022

(51) Int. Cl. A61M 1/30; A61M 1/16; B01J 20/18

(52) U.S. Cl. CPC A61M 1/301; A61M 1/1694; A61M 1/1603; B01J 20/18; A61M 1/1668; A61M 2205/3334; A61M 2205/3368

(57) ABSTRACT

The hemodialysis system with dialysate recycling uses a urea-adsorbing zeolite to remove urea from used dialysate, thus allowing the dialysate to be recycled. The hemodialysis system includes a housing and a dialyzer mounted on the housing. Similar to a conventional hemodialysis dialyzer, the dialyzer has blood inlet and blood outlet ports and dialysate inlet and dialysate outlet ports. The blood inlet port is adapted for receiving blood from the patient to be cleaned, and the blood outlet port is adapted for outputting cleaned blood, which is returned to the patient. A dialysate container may be mounted on the exterior of the housing and is adapted for receiving dialysate and the urea-adsorbing zeolite. Clean dialysate is fed from the dialysate container to the dialysate inlet port of the dialyzer, and used dialysate is recirculated from the dialysate outlet port of the dialyzer through the dialysate container.

### BACKGROUND

### 1. Field

The disclosure of the present patent application relates to hemodialysis, and particularly to a hemodialysis system with dialysate recycling that uses a urea-adsorbing zeolite to remove urea from the used dialysate, allowing the dialysate to be recycled.

### 2. Description of the Related Art

Hemodialysis (also sometimes referred to as simply “dialysis”) is a process of purifying the blood of a person whose kidneys are not working normally. Hemodialysis achieves the extracorporeal removal of waste products, such as creatinine, urea and free water, from the blood when the kidneys are in a state of kidney failure. Hemodialysis is a fluid mechanical process and can be performed as an outpatient or by inpatient therapy. Routine hemodialysis is usually conducted in a dialysis outpatient facility, which is typically either a purpose-built room in a hospital or a dedicated, stand-alone clinic. Less frequently, hemodialysis is performed at home. Hemodialysis involves a typically large and complex machine, which is why dialysis treatments in a clinic are far more common. In a clinic, hemodialysis is initiated and managed by specialized staff, whereas home dialysis requires training and practice in order to allow the process to be self-initiated and managed.

The principle of hemodialysis is the same as other methods of dialysis, i.e., it involves diffusion of solutes across a semipermeable membrane. Hemodialysis utilizes counter-current flow, where the dialysate is flowing in the opposite direction to blood flow in the extracorporeal circuit. Counter-current flow maintains the concentration gradient across the membrane at a maximum and increases the efficiency of the dialysis. Fluid removal is performed by ultrafiltration, and is achieved by altering the hydrostatic pressure of the dialysate compartment, causing free water and some dissolved solutes to move across the membrane along a created pressure gradient.

FIG. 2 illustrates a conventional prior art hemodialysis machine 100, which performs the hemodialysis process by pumping the patient's blood B and the dialysate D through the dialyzer 102. Typically, an extensive water purification system is absolutely critical for hemodialysis. Since dialysis patients are exposed to vast quantities of water, which is mixed with dialysate concentrate to form the dialysate, even trace mineral contaminants or bacterial endotoxins can filter into the patient's blood. Because the damaged kidneys cannot perform their intended function of removing impurities, ions introduced into the bloodstream via water can build up to hazardous levels, causing numerous symptoms or death. Thus, the fresh dialysate D, which is stored in dialysate tank 104, is made from highly purified water.

The dialyzer 102 is the piece of equipment that actually filters the blood B. Almost all dialyzers in use today are of the hollow-fiber variety. A cylindrical bundle of hollow fibers, whose walls are composed of a semi-permeable membrane, is anchored at each end into “potting compound”, which is a glue-like adhesive. This assembly is then placed into a clear plastic cylindrical shell 106 with four openings or ports. One opening at each end of the cylinder, such as ports 108 and 110, communicates with each end of the bundle of hollow fibers. This forms the “blood compartment” of the dialyzer 102. Two other ports 114, 116 are formed through the side of the cylinder 106. These communicate with the space around the hollow fibers, which is referred to as the “dialysate compartment.” Blood B is pumped via the blood ports 108, 110 through this bundle of very thin capillary-like tubes, and the dialysate D is pumped through the space surrounding the fibers. Pressure gradients are applied when necessary to move fluid from the blood to the dialysate compartment.

A conventional blood pump 118 drives the flow of blood B from the patient through port 108 and into the blood compartment of dialyzer 102. The cleaned blood CB is then driven out of port 110 and back into the patient. The pressure of the inflowing blood B may be measured by an arterial pressure monitor 120, and the pressure of the outflowing cleaned blood CB may be measured by a venous pressure monitor 122. For accuracy, the pressure of the inflowing blood B may also be measured just before injection into the dialyzer 102 by an inflow pressure monitor 126. During the diffusion process within the dialyzer 102, there is a possibility that small bubbles of air could enter the cleaned blood CB. Thus, a combination air trap and air detector 124 is typically employed to monitor and prevent any air bubbles being returned to the patient, which could be fatal.

The fresh dialysate D is pumped from the tank 104 by a conventional pump 128 through port 114 and into the dialysate compartment of the dialyzer 102. The used dialysate UD, which contains urea, creatinine, free water, and other waste products, such as potassium and phosphate, exits the dialysate compartment through port 116 and is stored in the used dialysate tank 130. In typical hemodialysis, the dialysate is not recycled. Thus, the presence of the two tanks 104 and 130 is necessary. Both tanks are typically relatively bulky and prevent conventional hemodialysis machines from being portable or even easily stored. Additionally, anticoagulants, such as heparin and the like, are typically injected into the blood just prior to the blood flowing into the dialyzer. The necessity for additional pumps, ports, and storage containers for the heparin also adds to the overall bulk of the hemodialysis machine.

A typical hemodialysis machine, such as that diagrammatically illustrated in FIG. 2, is about the size of a filing cabinet, and is usually permanently or semi-permanently maintained at the intended site of the procedure. Because of the bulk of such hemodialysis machines, transport, storage, and maintenance of the machines is very difficult. It would be desirable to be able to avoid the necessity for the additional storage tanks by effectively recycling the dialysate. Thus, a hemodialysis system with dialysate recycling solving the aforementioned problems is desired.

### SUMMARY

The hemodialysis system with dialysate recycling uses a urea-adsorbing zeolite to remove urea from used dialysate, thus allowing the dialysate to be recycled. The hemodialysis system with dialysate recycling includes a housing and a dialyzer mounted on the housing. A dialyzer holder may be secured to the housing, such that the dialyzer is removably disposed within the dialyzer holder, allowing the dialyzer to be temporarily removed for replacement, cleaning, transport, or storage.

Similar to a conventional hemodialysis dialyzer, the dialyzer has blood inlet and blood outlet ports and dialysate inlet and dialysate outlet ports. The blood inlet port is adapted for receiving blood from the patient to be cleaned, and the blood outlet port is adapted for outputting cleaned blood to be returned to the patient. A dialysate container may be mounted on the exterior of the housing and is adapted for receiving dialysate and a urea-adsorbing zeolite. Clean dialysate is fed from the dialysate container to the dialysate inlet port of the dialyzer, and used dialysate is recirculated from the dialysate outlet port of the dialyzer through the dialysate container. The dialyzer operates in a manner similar to that of a conventional dialyzer and may make use of counter-current flow, where the clean dialysate flowing into the dialyzer through the dialysate inlet port flows in the opposite direction to the blood flowing through the dialyzer. Counter-current flow maintains the concentration gradient across the semipermeable membrane inside the dialyzer at a maximum and increases the efficiency of the dialysis. Fluid removal from the patient's blood is performed by ultrafiltration, and is achieved by altering the hydrostatic pressure of the dialysate compartment within the dialyzer, causing free water and the dissolved solutes to move across the semipermeable membrane along the created pressure gradient.

As the used dialysate is circulated back through the dialysate container, the urea-adsorbing zeolite within the dialysate container adsorbs the urea from the used dialysate, allowing it to be recycled as clean dialysate, which is recirculated back through the dialyzer. It should be understood that any suitable type of urea-adsorbing zeolite may be used. An exemplary urea-adsorbing zeolite is Molecular Sieve 13X (Nas₈₆(AlO₂)₈₆(SiO₂)₁₀₆]·nH₂O).

Similar to a conventional hemodialysis system, a blood inlet tube is provided for carrying the blood from the patient to be cleaned to the blood inlet port of the dialyzer, a blood outlet tube is provided for carrying the cleaned blood from the blood outlet port of the dialyzer to the patient, a dialysate inlet tube is provided for carrying the clean dialysate from the dialysate container to the dialysate inlet port of the dialyzer, and a dialysate outlet tube is provided for recirculating the used dialysate from the dialysate outlet port of the dialyzer to the dialysate container. A first pump selectively and controllably drives the blood from the patient to be cleaned through the blood inlet tube, and a second pump selectively and controllably drives the clean dialysate through the dialysate inlet tube.

An air bubble sensor may be mounted on the housing for monitoring the cleaned blood carried by the blood outlet tube for the presence of air bubbles, and a temperature sensor may also be mounted on the housing for measuring a temperature of the cleaned blood carried by the blood outlet tube. A heater may also be provided for selectively raising the temperature of the cleaned blood carried by the blood outlet tube. Additionally, a flow rate sensor may also be mounted on the housing for selectively monitoring the flow rate of the cleaned blood carried by the blood outlet tube.

These and other features of the present subject matter will become readily apparent upon further review of the following specification and drawings.

### BRIEF DESCRIPTION OF THE DRAWINGS

FIG. 1 is a diagrammatic front view of a hemodialysis system with dialysate recycling.

FIG. 2 is a diagram of a conventional prior art hemodialysis machine.

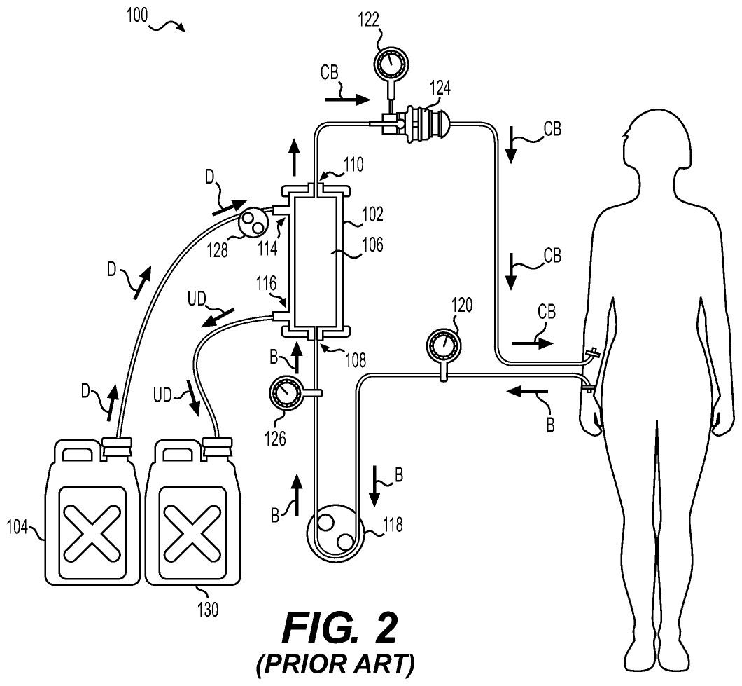

FIG. 3 is a block diagram of the hemodialysis system with dialysate recycling of FIG. 1.

FIG. 4 is a plot of measured concentrations of urea adsorbed by a zeolite from a dialysate sample as a function of time for differing masses of the zeolite, and also comparing unstirred zeolite/dialysate suspensions against a stirred zeolite/dialysate suspension.

FIG. 5 is a plot of removal efficiency of the urea adsorption by the zeolite from the dialysate sample of FIG. 4 for the differing masses of the zeolite, and also comparing unstirred zeolite/dialysate suspensions against the stirred zeolite/dialysate suspension.

Similar reference characters denote corresponding features consistently throughout the attached drawings.

### DETAILED DESCRIPTION OF THE PREFERRED EMBODIMENTS

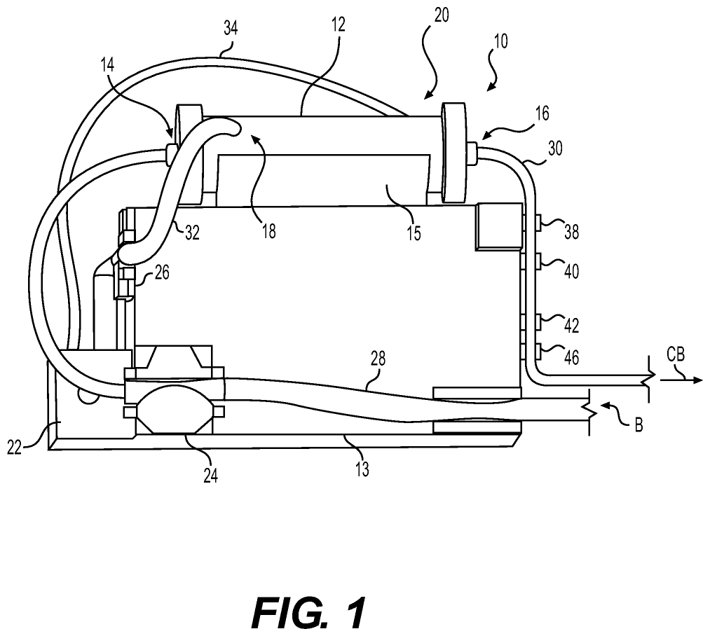

The hemodialysis system with dialysate recycling, designated generally as 10 in the drawings, uses a urea-adsorbing zeolite to remove urea from used dialysate, thus allowing the dialysate to be recycled. As shown in FIG. 1, the hemodialysis system 10 includes a housing 13 and a dialyzer 12 mounted on the housing 13. A dialyzer holder 15 may be secured to the housing 13, such that the dialyzer 12 is removably held within the dialyzer holder 15, allowing the dialyzer 12 to be temporarily removed for replacement, cleaning, transport, or storage. It should be understood that the overall configuration and dimensions of the housing 13 and the dialyzer holder 15 are shown in FIG. 1 for exemplary purposes only. Since the recycling of the dialysate removes the need for large storage tanks, the hemodialysis system with dialysate recycling 10 may be used as a portable dialysis system. The housing 13 may be dimensioned for portability and home use. For example, the housing 13 may be configured as a box with dimensions of 35 cm×40 cm×25 cm. Additionally, it should be understood that any suitable type of hemodialysis dialyzer may be used. For example, the dialyzer 12 may use a polyethersulfone membrane to separate the dialysis compartment from the blood compartment within the housing of the dialyzer 12.

Similar to a conventional hemodialysis dialyzer, the dialyzer 12 has blood inlet and blood outlet ports 14, 16, respectively, and dialysate inlet and dialysate outlet ports 18, 20, respectively. The blood inlet port 14 is adapted for receiving blood B from the patient to be cleaned, and the blood outlet port 16 is adapted for outputting cleaned blood CB to be returned to the patient. A dialysate container 22 may be mounted on the exterior of the housing 13 and is adapted for receiving dialysate D and a urea-adsorbing zeolite. It should be understood that the overall dimensions and configuration of the dialysate container 22 are shown in FIG. 1 for exemplary purposes only.

Clean dialysate D is fed from the dialysate container 22 to the dialysate inlet port 18 of the dialyzer 12, and used dialysate UD is recirculated from the dialysate outlet port 20 of the dialyzer 12 through the dialysate container 22. The dialyzer 12 operates in a manner similar to that of a conventional dialyzer and may make use of counter-current flow, where the clean dialysate D flowing into the dialyzer 12 through the dialysate inlet port 18 flows in the opposite direction to the blood B flowing through the dialyzer 12. Counter-current flow maintains the concentration gradient across the semipermeable membrane inside the dialyzer 12 at a maximum and increases the efficiency of the dialysis. Fluid removal from the patient's blood B is performed by ultrafiltration, and is achieved by altering the hydrostatic pressure of the dialysate compartment within the dialyzer 12, causing free water and the dissolved solutes to move across the semipermeable membrane along the created pressure gradient.

As the used dialysate UD is circulated back through the dialysate container 22, the urea-adsorbing zeolite within the dialysate container 22 adsorbs the urea from the used dialysate UD, allowing it to be recycled as clean dialysate D, which is recirculated back through the dialyzer 12. It should be understood that any suitable type of urea-adsorbing zeolite may be used. An example of such a urea-adsorbing zeolite is the Molecular Sieve 13X (Na₈₆[(AlO₂)₈₆(SiO₂)₁₀₆]·nH₂O) zeolite. The urea-adsorbing zeolite may be, for example, in the form of a powder, and a mixer or stirrer 52 may be disposed within, or attached to, the dialysate container 22 to maintain a homogeneous mixed suspension of dialysate and zeolite powder within the dialysate container 22.

In order to test the efficacy of molecular sieve 13X in removing urea, urea concentration in a sample dialysate was measured, both before and after addition of Molecular Sieve 13X zeolite. Using the non-linear multiple regression test, the results showed that as the mass of the zeolite adsorbent added to the sample dialysate increased, the urea concentration decreased by almost 0.18 g/mol. Based on these results, it was found that the required mass of the adsorbent over a period of time estimated for a normal human male with no other related diseases is 275 g of zeolite for each 1 liter of dialysate. This calculation is based on the flow required for each patient, where the typical blood flow rate is estimated to be in the range of 250-400 mL/min, while for the dialysate, the estimated flow rate is 500-800 mL/min. As a result, the dialysate must be twice the volume of blood to be purified. Thus, through the addition of the zeolite, far less dialysate must be used than in conventional hemodialysis. The addition of the adsorbent to the diffusion and ultrafiltration process also accelerates the purification of the blood, reducing the average time for hemodialysis to between one and two hours per session.

With regard to the other components removed from the blood, the various component concentrations in the dialysate may be adjusted relative to those of normal plasma and/or the uremic fluid. Table 1 below shows exemplary desired ratios to be used for each component to be removed from a patient's blood. The figures in Table 1 are based on an adult human male with kidney failure, but no additional conditions or diseases. As an example, in Table 1, in order to maintain glucose at 100 mEq/L, the diffusion process must be stopped, requiring the dialysate to have the same glucose concentration. As another example, the calcium concentration in the uremic fluid is 2 mEq/L but the desired concentration in normal plasma is 3 mEq/L. Thus, the dialysate should be prepared to have 4 mEq/L in order to achieve proper purification of the patient's blood.

TABLE 1
Dialysate Ratios for Blood Component Removal

| | Normal Plasma | Uremic fluid | Dialysate |
|---|---|---|---|
| Component | (mEq/L) | (mEq/L) | (mEq/L) |
| Na⁺ | 142 | 142 | 142 |
| K⁺ | 5 | 7 | 3 |
| Ca⁺⁺ | 3 | 2 | 4 |
| Mg⁺⁺ | 1.5 | 1.5 | 1.5 |
| Cl⁻ | 107 | 107 | 107 |
| HCO₃⁻ | 24 | 14 | 34 |
| Lactate⁻ | 1.2 | 1.2 | 1.2 |
| HPO₄⁻ | 3 | 9 | 0 |
| Urate⁻ | 0.3 | 2 | 0 |
| Sulfate⁻ | 0.5 | 3 | 0 |
| Glucose | 100 | 100 | 100 |
| Urea | 26 | 200 | 0 |
| Creatinine | 1 | 6 | 0 |

Similar to a conventional hemodialysis system, a blood inlet tube 28 is provided for carrying the blood B from the patient to be cleaned to the blood inlet port 14 of the dialyzer 12, a blood outlet tube 30 is provided for carrying the cleaned blood CB from the blood outlet port 16 of the dialyzer 12 to the patient, a dialysate inlet tube 34 is provided for carrying the clean dialysate D from the dialysate container 22 to the dialysate inlet port 18 of the dialyzer 12, and a dialysate outlet tube 32 is provided for recirculating the used dialysate UD from the dialysate outlet port 20 of the dialyzer 12 to the dialysate container 22. A first pump 24 selectively and controllably drives the blood B from the patient to be cleaned through the blood inlet tube 28 to the blood compartment of the dialyzer 12, and back to the patient through the blood outlet tube 30. A second pump 26 selectively and controllably recirculates the used dialysate UD from the dialysate compartment of the dialyzer 12 back to the dialysate container 22 through the dialysate outlet tube 32, and the clean dialysate D is recycled in dialysate container 22 back to the dialysate compartment through the dialysate inlet tube 34.

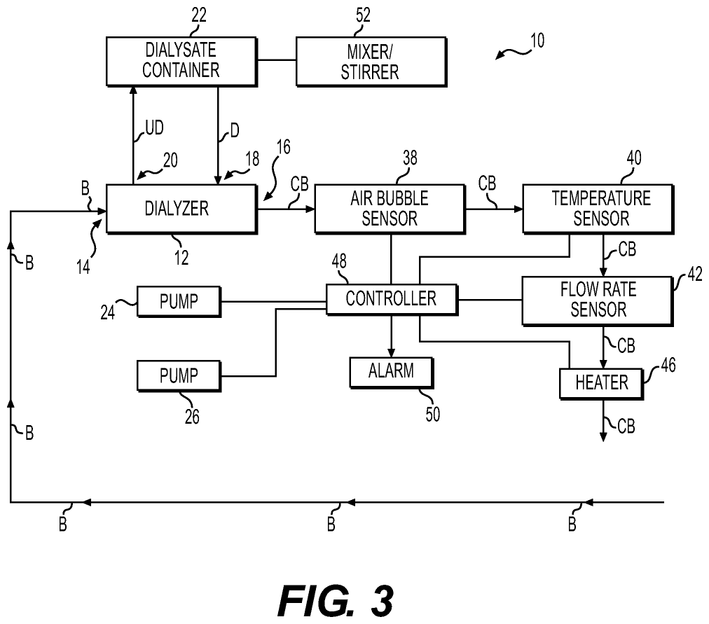

As shown in FIG. 3, the first pump 24 and the second pump 26 may be in communication with a controller 48, which may be programmed and/or operated manually to control the action of each of pumps 24, 26. It should be understood that controller 48 may be any suitable type of controller, such as a microprocessor, a programmable logic controller, logic circuitry, a personal computer, a microcontroller, or the like. Controller 48 may be contained within housing 13 or may be externally or remotely located with respect to housing 13.

An air bubble sensor 38, also in communication with controller 48, may be mounted on the housing 13 for monitoring the cleaned blood carried by the blood outlet tube 30 for the presence of air bubbles. It should be understood that any suitable type of air bubble sensor may be utilized. If air bubble sensor 38 detects the presence of air bubbles in the cleaned blood CB, the controller 48 may actuate a visible and/or audio alarm 50 to warn a technician or user and/or stop pumps 24 and 26 from operating to prevent the cleaned blood from flowing back to the patient.

A temperature sensor 40 in communication with the controller 48 may also be mounted on the housing 13 for measuring the temperature of the cleaned blood CB carried by the blood outlet tube 30. If the cleaned blood has a temperature below a pre-set threshold, controller 48 may actuate the alarm 50 and/or stop the pumps 24 and 26 from operating to prevent the cleaned blood CB from flowing back to the patient. Alternatively, a heater 46, also in communication with controller 48, may be provided for selectively raising the temperature of the cleaned blood CB carried by the blood outlet tube 30 to the desired threshold temperature. For example, the threshold temperature may be set to 37° C., the average body temperature of an adult human. Additionally, a flow rate sensor 42 in communication with the controller 48 may also be mounted on the housing 13 for selectively monitoring the flow rate of the cleaned blood CB carried by the blood outlet tube 30.

By monitoring and controlling both temperature and fluid flow rate, there is no need to use anticoagulants, such as heparin, in the hemodialysis system 10 with dialysate recycling. Anticoagulation is achieved through maintaining the temperature constant at the 37° C. threshold with a high rate of flow. For example, the blood flow rate may be set at approximately 250-300 mL/min. Based on the measurements from temperature sensor 40 and/or flow rate sensor 42, if the controller 48 determines that there is a low level of risk to the patient, the alarm 50 may be actuated and the patient may be given an additional anticoagulant, for example. If the controller 48 determines that there is a high risk to the patient, the blood flow can be stopped. Further, since low temperature blood returning to the patient can cause harm to the patient, the heater 46 can be actuated accordingly. Heater 46 can be controlled by the controller 48 to maintain a constant temperature of the cleaned blood CB at, for example, 37° C.

As discussed above, a stirrer/mixer 52 may be disposed in the dialysate container 22 to maintain a homogeneous suspension of the zeolite. In order to test the efficacy of stirring, an experiment was performed to test urea concentration in a sample dialysate when masses of 9 g, 12 g and 15 g of the zeolite were added, where there was no stirring, and also when a mass of 11 g of zeolite was added, but with stirring. Table 2 shows the results of measured concentrations of urea measured between a start time (shown as 0 minutes) and an end time of 120 minutes.

TABLE 2
Urea Concentrations Following Adsorption

| | Urea | Urea | Urea | Urea |
|---|---|---|---|---|
| | concentration | concentration | concentration | concentration |
| | (mg/dL) for 9 g | (mg/dL) for 12 g | (mg/dL) for 15 g | (mg/dL) for 11 g |
| Time | of zeolite, | of zeolite, | of zeolite, | of zeolite, |
| (min.) | not stirred | not stirred | not stirred | with stirring |
| 0 | 19 | 19 | 19 | 19 |
| 15 | 14 | 10 | 9.087 | 7.917 |
| 30 | 10 | 9 | 8.261 | 6.333 |
| 45 | 9 | 8 | 7.435 | 4.750 |
| 60 | 8 | 7 | 6.609 | 3.167 |
| 120 | 8 | 7 | 6.609 | 3.167 |

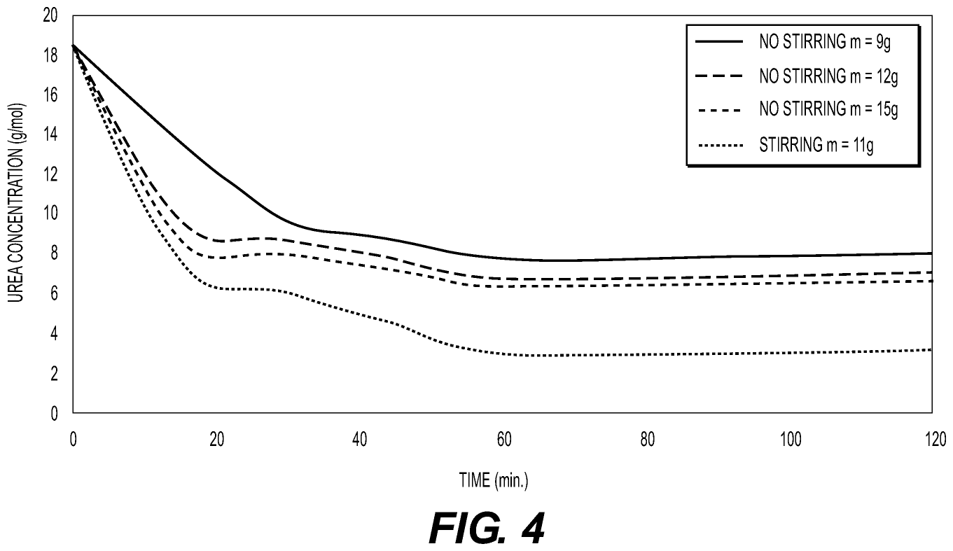

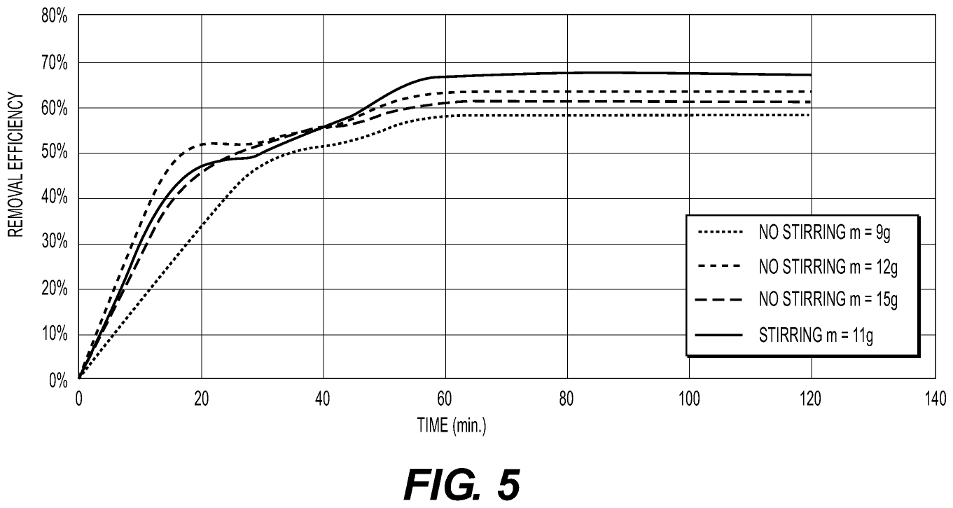

It can be seen in Table 2 above that the urea concentration not only decreases with addition of more zeolite, but the stirring of the zeolite has a significant effect on the adsorption of the urea. These results are also plotted in FIGS. 4 and 5, where FIG. 4 shows the measured urea concentration as a function of time, and FIG. 5 shows the corresponding removal efficiency as a function of time.

It is to be understood that the hemodialysis system with dialysate recycling is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A home-use dialysis system providing dialysate recycling in a portable device with dimensions of a medium-sized box of 35×40×25 cm, comprising:
a housing containing electrical circuits that have light and compact components;
a dialyzer mounted on the housing, the dialyzer having a blood inlet port and a blood outlet port, the blood inlet port being adapted for receiving uremic blood from a patient, and the blood outlet port being adapted for outputting cleaned blood to be returned to the patient, the dialyzer further having a dialysate inlet and a dialysate outlet;
a dialysate container adapted for receiving dialysate, the dialysate container being connected to the dialysate inlet and dialysate outlet ports;
a urea-adsorbing zeolite disposed in the dialysate container whereby clean dialysate is fed from the dialysate container to the dialysate inlet port of the dialyzer, and used dialysate is recirculated from the dialysate outlet port of the dialyzer through the dialysate container for removal of urea by the urea-adsorbing zeolite to provide clean dialysate, the dialysate container containing a blade to ensure continuous mixing of the dialysate and the urea-adsorbing zeolite in order to maintain a homogeneous suspension;
an alarm system connected to a hospital to provide notification in case of complications;
two pumps including a first pump connected to a blood inlet tube for driving the blood from the patient to be cleaned with 250-300 L/min as a speed;
a second pump connected to the dialysate inlet for driving the clean dialysate through the dialyzer with 500-600 L/min as a speed; and
an air bubble sensor located in a blood outlet for monitoring the cleaned blood carried by a blood outlet tube for the presence of air bubbles, wherein when an air bubble is detected the hemodialysis system turns off immediately.

2. The home-use hemodialysis system as recited in claim 1, further comprising: a dialyzer holder secured to the housing, the dialyzer holder having a horizontal orientation to avoid gravity effects during movement and ensure minimal variations oof a circulated speed, the dialyzer being removably held within the dialyzer holder.

3. The home-use hemodialysis system as recited in claim 1, wherein the dialysate has a composition of components to ensure a complete dialysis session without need for different adsorbents, the components having respective concentrations as follows: 142 mEq/L of Na+, 3 mEq/L of K+, 4 mEq/L of Ca++, 1.5 mEq/L of Mg++, 107 mEq/L of Cl—, 34 mEq/L of HCO3—, 1.2 mEq/L of Lactate, 0 mEq/L of HPO4—, 0 mEq/L of urate, 0 mEq/L of sulfate —, and 100 mEq/L of glucose.

4. (canceled)

5. (canceled)

6. The home-use hemodialysis system as recited in claim 1, further comprising a heater that maintains temperature at 37° C., and a temperature sensor located in the blood outlet tube that turns off the hemodialysis system of less than 35° C. are detected,

7. (canceled)

8. The home-use hemodialysis system as recited in claim 6, further comprising a flowrate sensor in the blood outlet for selectively monitoring a flowrate of cleaned blood carried by the blood outlet tube and as a result monitoring turning the alarm system on when the flowrate is less than 300 mL/min for the patient to consume anticoagulation pills, turning the alarm system off when the flowrate is more than 300 mL/min, and turning the home-use hemodialysis system off when the flowrate is less than 250 mL/min.

9. The home-use hemodialysis system as recited in claim 1, further using a molecular sieve 13X zeolite that is distributed in a dialysate solution including a mass of
